(19) United States
(12) Patent Application Publication
(10) Pub. No.: US 2006/0264907 A1
(43) Pub. Date: Nov. 23, 2006

(54) Catheters having stiffening mechanisms

(75) Inventors: Joseph Eskridge, Clyde Hill, WA (US); Gilbert Clarke, Seattle, WA (US)

Correspondence Address: SPECKMAN LAW GROUP PLLC, 1201 THIRD AVENUE, SUITE 330, SEATTLE, WA, 98101, US

(73) Assignee: Pulsar Vascular, Inc., Seattle, WA (US)

(21) Appl. No.: 11/224,276

(22) Filed: Sep. 12, 2005

Related U.S. Application Data
(60) Provisional application No. 60/676,925, filed on May 2, 2005.

(51) Int. Cl. A61M 25/00

(52) U.S. Cl. 604/528

(57) ABSTRACT

Catheters having selectively insertable or selectively activatable and releasable stiffening mechanisms are provided. In general, the catheter is inserted, navigated and withdrawn from a subject in a relaxed, flexible condition and stiffening mechanisms are deployed to prevent the catheter from shifting during placement or operation of an accessory device or tool through the catheter. Stiffening members(s) may be inserted into and removed from one or more longitudinal channel(s) provided in proximity to the catheter wall and generally coaxial with the primary catheter lumen to change the stiffness properties of the catheter. The properties, configuration and size of the stiffening members and channels may be varied to vary the stiffness properties of the catheter and stiffening members may be constructed from materials having shape change properties or materials that change conformation or stiffness with application of heat, current or electrical field. Stiffening mechanisms may also employ energy absorbing and viscoelastic polymer materials having variable stiffness properties depending on ambient conditions.

### CROSS-REFERENCE TO RELATED APPLICATION

This application claims priority under 35 U.S.C. 119(e) to U.S. Patent Application No. 60/676,925, filed May 2, 2005.

### TECHNICAL FIELD OF THE INVENTION

The present invention relates to catheters having stiffening mechanisms that are selectively activatable and releasable by an operator to increase the stiffness or flexibility of a catheter or a portion of a catheter before, during or after guidance and placement of the catheter.

### BACKGROUND OF THE INVENTION AND DESCRIPTION OF PRIOR ART

Catheters are flexible tubes used for navigating internal body vessels and lumens and guiding devices within the body, such as in the vasculature, urinary tract, spinal column, fallopian tubes, bile ducts, and the like, and are often used in connection with minimally invasive diagnostic or surgical techniques. Catheters may be used for medical procedures to examine, diagnose and treat internal conditions while positioned at a target location within the body that is otherwise inaccessible. An intravascular catheter is generally inserted and advanced through a valved introducer fitting into a blood vessel near the surface of the body, such as the femoral artery, and is guided through the vasculature to a desired location. Catheters are used for coronary vascular and cardiac-related interventional operations, as well as neurovascular interventions, peripheral vascular, renal, and other types of intravascular interventions. Medical devices and instruments may be guided, through the catheter, to the desired site and operated.

Guiding catheters are generally the introductory catheters through which various interventional devices and instruments are supported and guided during passage to a target internal location. Guide catheters are sold in a variety of pre-formed sizes and shapes, customized for desired procedures. Microcatheters are generally smaller diameter catheters used for delivery of agents, devices or instruments through small diameter vessels in neurovascular interventions. Therapeutic devices and agents such as embolization coils, pharmaceutical agents, and embolic materials are delivered to a neurovascular site through microcatheters, for example. Both flow-guided and wire-guided microcatheters are used for interventional navigation. Catheters intended for use in neurovascular applications often have soft, shaped distal tips intended to improve navigation to and retention in particular neurovascular sites.

Intravascular catheters must be flexible enough to navigate through the sometimes tortuous vasculature without damaging tissue, yet stiff enough to provide “pushability” through the vasculature and support for internally guided medical devices, fluids and instruments, and must be kink-resistant. Guide catheters may have a composite construction that provides greater stiffness and support in proximal areas with more softness and flexibility in distal areas. Variable stiffness along the length of a guide catheter is typically provided by varying the construction and/or diameter and/or wall thickness and/or material along the length of the catheter. There are many examples of catheters having variable stiffness along their lengths in the prior art literature.

It may also be useful for an operator to have the ability to vary the stiffness and/or conformation of a catheter over time so that a guide catheter, for example, may be adjusted to provide different stiffness properties and/or conformations during and after placement of the catheter at a target site. U.S. Pat. No. 4,248,234, for example, describes a catheter having variable flexibility/stiffness properties over time to provide a high degree of stiffness during placement and a lower degree of stiffness after placement. Variable stiffness is achieved using a second, off-axis lumen that can be controllably pressurized by filling with fluid.

U.S. Pat. No. 6,663,648 discloses a balloon dilatation catheter having a transition assembly positioned between the proximal cannula and the distal end section with a floating stiffening member retained in the transition assembly.

U.S. Pat. No. 5,334,168 discloses the use of nitinol memory elements selectively activated by current to deform the distal end of a catheter and thereby guide it to a desired site. This patent describes several ways to use temperature/current-activated memory elements used in connection with a catheter to change the profile of the catheter at its distal end.

U.S. Pat. No. 4,909,787 discloses a guide catheter having variable, operator-controlled flexibility at the distal end to increase stiffness of the distal end of the catheter after placement. The stiffener is an eccentrically positioned fluid-filled chamber.

PCT International Publication WO 02/078777 discloses a variable stiffness heating catheter providing variations in stiffness along the length of the catheter shaft. The purpose is to provide higher stiffness, hence pushability at the proximal end with a higher flexibility, more maneuverable distal end.

One of the problems encountered with intravascular catheters, and particularly guide catheters, is movement or “kicking” of the catheter following placement and movement or “kicking” of the catheter while advancing other catheters or accessory devices and instrument through the guide catheter. In some cases, shifting and adjustment of the guide catheter during an intervention may require withdrawal of the accessory device or instrument and replacement of the guide catheter, which prolongs the intervention and thereby increases the risk to the patient. Shifting of the guide catheter during an intervention may also damage vessel walls or produce shifting of the interventional accessory device or instrument, producing internal injury. Various types of positioning and stiffening schemes have been conceived to stabilize placement of catheters.

U.S. Pat. No. 6,146,339 discloses a guide wire having operator controllable tip stiffness using fluid filled balloons. The guidewire may inserted and withdrawn with the balloon(s) in a flexible, deflated condition and the balloon(s) may be filled to expand and stiffen the distal section of the guidewire to aid in positioning and retaining the guidewire in the desired location.

U.S. Pat. No. 5,531,685 discloses a flexible tubular member catheter having a shape memory element that's selectively heated using current, injection of fluids, RF induction or IR irradiation to change the shape/configuration and increase the axial and bending stiffness of the catheter to hold or wedge the catheter in place as operations are conducted using the catheter. The catheter has a multi-layer construction with the shape memory element in a generally tubular configuration coaxial with the catheter and lumen. The purpose is to enable local changes in stiffness upon command, providing added support as other objects are passed through the catheter lumen.

PCT International Publication WO 02/087690 discloses a catheter system that has an extending, expandable distal coil (shape memory metal) that extends and expands to wedge the coil in a vessel and implant a lead.

PCT International Publication WO 2004/105599 discloses catheters having intra-catheter stiffener elements that temporarily stiffen the catheter lumen and/or tip independently. The specific application is a multi-lumen, split-tip hemodialysis catheter for use with multiple guidewires.

### SUMMARY OF THE INVENTION

The present invention is directed to catheters having stiffening mechanisms that are selectively insertable, or selectively activatable and releasable, by an operator, to vary the stiffness properties of a catheter, or a portion of a catheter, at different times during use of the catheter. For many applications, the objective is to provide a catheter that has sufficient flexibility and pushability at the time of insertion and withdrawal of the catheter to facilitate navigation to and from a target site and to provide a stiffer catheter, or a stiffer portion of the catheter, during placement or operation of another device or instrument through the catheter.

In one embodiment of a catheter of the present invention, at least one longitudinal channel is provided in proximity to a wall of the catheter, such as along the outer wall of a catheter, the longitudinal channel being generally coaxial with the primary catheter lumen. A stiffening member, such as a wire or rod, is provided and may be inserted into and removed from the channel by an operator to change the stiffness of the catheter from a more flexible catheter (when the stiffener is not inserted) to a stiffer catheter (when one or more stiffeners are inserted). The properties, configuration and size of the longitudinal channel(s) and the stiffener wire(s) may be varied to vary the stiffness properties of the catheter. In this embodiment, a catheter kit comprises a catheter having at least one longitudinal channel and at least one stiffening member sized for insertion into and removal from at least one channel.

The longitudinal channel(s) and stiffener(s) may extend substantially the full length of the catheter or may extend for only a portion of the length of the catheter. In one embodiment, the channel(s) and complementary stiffener(s) extend for substantially the entire length of the catheter and terminate in proximity to the distal end of the catheter. In another embodiment, the channel(s) and complementary stiffener(s) extend for a length of the catheter that is approximately 5-15 cm proximal to the distal end of the catheter. According to yet another embodiment, channel(s) and complementary stiffener(s) extend for a length of about 10-50 cm starting at the proximal end of the catheter. In an alternative embodiment, one or more channel(s) may have a distal portal that permits the stiffener to extend out of the channel. In this embodiment, the stiffener may serve as a primary or secondary guidewire or lead.

One or more longitudinal channels may be provided having a proximal portal for introduction of one or more stiffening members. In general, the channels are provided on or in proximity to the outer surface of the catheter wall and the catheter wall may form an integral part of the channel. Although channels provided on or in proximity to the outer surface of the catheter wall are preferred for many applications, channels may be provided as lumens within the wall of the catheter in alternative embodiments. In some embodiments, the channel may traverse the catheter wall so that a portion of the channel is on or in proximity to the outer catheter wall and a portion of the channel is on or in proximity to an inner catheter wall or within a primary or secondary catheter lumen.

For many applications, the channel(s) have a longitudinal axis that is generally aligned and coaxial with the longitudinal axis of the catheter. For applications in which the diameter of the outer catheter wall narrows toward the distal end of the catheter, the lontigudinal axis of the channel is aligned at a narrow angle to longitudinal axis of the catheter lumen. In alternative embodiments, the longitudinal axis of the channel(s) may be curved or provided in a shallow helical configuration that bends entirely or partially around the circumference of the catheter.

In one embodiment, at least two longitudinal channels are provided and are radially separated from one another by about 45-180°. In another embodiment, two longitudinal channels are provided and are radially separated from one another by about 60-115°, in another embodiment separated by approximately 80-100°. Multiple channels may be provided in a radially symmetrical or asymmetrical configuration, depending on the desired stiffening properties and locations. Multiple channels may have different channel conformations and/or different sizes and/or different lengths for use with different sizes and types of stiffeners. A lubricious layer or coating may be provided on an inner channel wall and/or on the outer surface of stiffener(s).

Stiffening elements may be provided as elements separate from the catheter for insertion into one or more channels in the catheter, as described above. These types of stiffening elements may be constructed, for example, from nitinol and other types of metallic and alloy wires. The stiffeners may have a constant or variable diameter and/or cross-sectional configuration over their length, may be constructed from different materials over their length and may have variable stiffness over their length. The distal tip(s) of the stiffener(s) are generally atraumatic and may have a specialized conformation.

In alternative embodiments, one or more stiffener element(s) may be pre-loaded and incorporated in one or more channel(s) in a catheter as an integrated assembly. Prior to insertion of the catheter during an intervention, one or more of the stiffener(s) may be withdrawn or partially withdrawn from the channel(s) to reduce the stiffness of the catheter during insertion and may be reinserted into the channel(s) following placement of the catheter to stiffen the catheter and reduce movement of the catheter during advancement of other catheters or accessory devices through the catheter. When multiple stiffeners having different stiffness properties are provided in multiple channels, the medical professional may adjust the stiffness of the catheter during insertion of the catheter, insertion and use of accessory devices, and during withdrawal of the catheter, by selectively withdrawing and inserting stiffener(s) having different stiffness properties.

In yet another embodiment, stiffener elements formed from a shape memory material or another material that changes conformation upon a change in the environment may be pre-loaded and incorporated in channels or recesses of a catheter, or embedded in or mounted on a catheter of the present invention. Stiffener elements may be constructed, for example, from materials having shape memory properties or other types of materials that are treatable to have different stiffness properties upon application of heat, current, electrical field, magnetic field or the like. In these embodiments, the pre-loaded stiffener elements are in a relaxed condition and conform generally to the configuration and axial alignment of the catheter at ambient conditions and are selectively activatable during transit of a catheter or following placement of a catheter to adopt a desired stiffness or shape upon application of heat, electrical or magnetic field, current, or the like.

In still another embodiment, channel walls or stiffener elements may incorporate a material such as an energy absorbing polymer material having variable stiffness properties depending on ambient conditions. Energy absorbing and viscoelastic polymers and polymer matrices are stiff and have high tensile strength upon application of force such as an impact or vibration and are soft and flexible in the absence of such force. In this embodiment, an energy absorbing or viscoelastic polymer that is in a soft and flexible condition at ambient body temperatures and pressures may be incorporated in one or more channel walls or pockets or recesses in the catheter wall. Following insertion and desired placement of the catheter, a stiffening force, such as vibration, may be applied to the energy absorbing polymer material, such as by insertion and vibration of a rod or another element within a channel, to stiffen the material and the catheter and prevent movement of the catheter relative to vessels during the use of accessory devices. When the stiffening force is withdrawn, the energy absorbing polymer material and the catheter become flexible and may be conveniently withdrawn. Energy absorbing polymers as well as nanocomposites and mesocomposites that are relatively rigid upon application of a force and are lightweight and flexible in absence of the force are suitable.

Pre-loaded stiffener elements may be provided in longitudinal configurations and channels or in other, three-dimensional configurations. Such stiffener elements may be embedded in or mounted on or provided in recesses within a catheter side wall and may be provided in discontinuous, annular, curved or serpentine shapes that are selected and placed at predetermined locations on the catheter to produce desired curves or other conformations at desired locations along the catheter that assist in stabilizing the catheter following placement.

Numerous catheter materials and constructions are known, and catheters of the present invention may be constructed using any materials, composite arrangements and conformations and construction techniques known in the art. Many catheters, for example, have a multi-layer construction and may be reinforced in sections or along their length, and may have different properties and dimensions along their length. Inner and/or outer surfaces may be provided with coatings or constructed from materials that enhance lubricity. Suitable coatings and materials are well known in the art. Radio-opaque markers may be incorporated in the catheters to allow for visualization and precise positioning, as is known in the art.

### BRIEF DESCRIPTION OF THE DRAWINGS

Various aspects of applicants' claimed inventions are illustrated schematically in the accompanying drawings, which are intended for illustrative purposes only and are not drawn to scale.

FIG. 1 shows a side perspective view of a proximal section of a catheter of the present invention having two channels for receiving rod-like stiffening elements.

FIG. 2A shows a proximal end view of the catheter section of FIG. 1 having two longitudinal channels for receiving rod-like stiffening elements.

FIG. 2B shows a proximal end view of another catheter of the present invention having two longitudinal channels for receiving rod-like stiffening elements.

FIG. 2C shows a proximal end view of another catheter of the present invention having two longitudinal channels for receiving rod-like stiffening elements provided in the catheter wall.

FIG. 3 shows a cross-sectional view of a distal portion of a catheter of the present invention having a channel for receiving a stiffening element that terminates proximal to the distal tip of the catheter.

FIG. 4 shows a cross-sectional view of a catheter of the present invention having a channel for receiving a stiffening element that traverses the catheter side wall from an outer surface of the catheter to an inner catheter surface and has a distal portal allowing a stiffener to extend from the distal tip of the catheter.

### DETAILED DESCRIPTION OF THE INVENTION

Catheters of the present invention comprise generally tubular structures having a substantially continuous side wall forming a lumen and may be used for a variety of purposes. Catheters of the present invention may be employed, for example, as guide catheters, delivery catheters, diagnostic catheters, angiographic catheters, infusion catheters, drug delivery catheters, ablation catheters, angioplasty or balloon angioplasty catheters, dilation catheters, stent catheters, neurovascular microcatheters, or the like, for delivery of accessory devices, instruments, pharmaceuticals or other agents, or the like, to a target site within the body that is generally accessible through the vasculature or a body opening or lumen. Catheters of the present invention may also include sheaths and other types of tubular structures used for delivery of devices, instruments, and agents to target sites within the body.

As used herein, the term “proximal” refers to a direction toward an operator and the site of catheter introduction into a subject along the path of the catheter system, and “distal” refers to the direction away from the operator and introduction site along the path of the catheter system toward a terminal end of the catheter assembly.

Many different catheter types and constructions are known in the art and catheters of the present invention may have a variety of constructions, properties, and the like. Catheters may, for example, comprise a multi-layer construction in which flexible tubing is reinforced with stiffer materials such as helical coils and braided materials to provide different stiffness properties along the length of the catheter. Coatings may be provided on the inner or outer surfaces of catheters to improve lubricity. Hydrophilic coatings are often provided on exterior surfaces to facility guidance through tortuous vasculature. Liners comprising lubricious materials such as fluoropolymer resins, films and coatings, such as TEFLON® PTFE and similar materials may be provided on inner catheter surfaces to enhance passage of accessory devices and systems through the catheter.

Catheters may also have different cross-sectional dimensions and/or thicknesses and/or flexibilities along their lengths. In general, catheters have a larger cross-sectional outer dimension and have thicker, less flexible walls in proximal sections and a smaller cross-sectional outer dimension with thinner, more flexible walls in distal sections. The length of a guide catheter may be up to 100 cm or more, and the outer lumen diameter (French size) of a guide catheter may range from 4-10F. The length of a microcatheter for use, for example, in neurovascular applications, may be up to 170-200 cm, and the outer lumen diameter of a microcatheter may range from about 1.5-3.5F. Other types of catheters are provided in generally standard lengths and diameters and may incorporate a flexible distal tip for a length of from about 5 cm to 50 cm from the terminal distal end that is soft, perhaps shapeable, and has one or more radiopaque markers for relative positioning by the physician. The tip configuration may be preformed or formable in a variety of configurations, including linear and curved, as well as angled. Catheters of the present invention may incorporate any of these features. U.S. Pat. Nos. 6,672,338, 6,152,944, 6,824,553, 6,863,678, 6,740,073, 6,626,889 and 6,679,836 are incorporated by reference herein in their entireties and disclose exemplary types of catheters and catheter constructions, any of which may be used in connection with catheters of the present invention. Numerous other catheter types and constructions are known in the art and may be used in combination with the novel catheter features described herein.

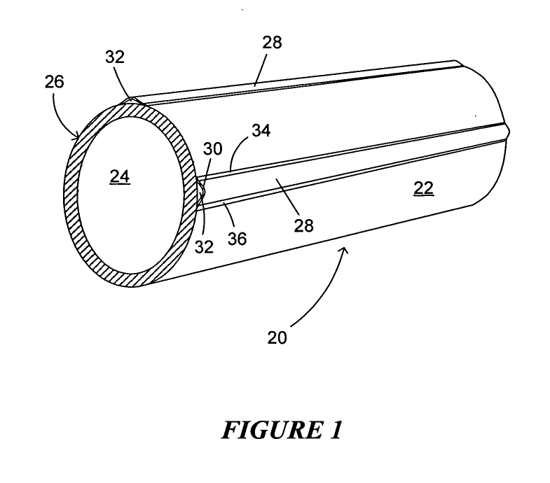

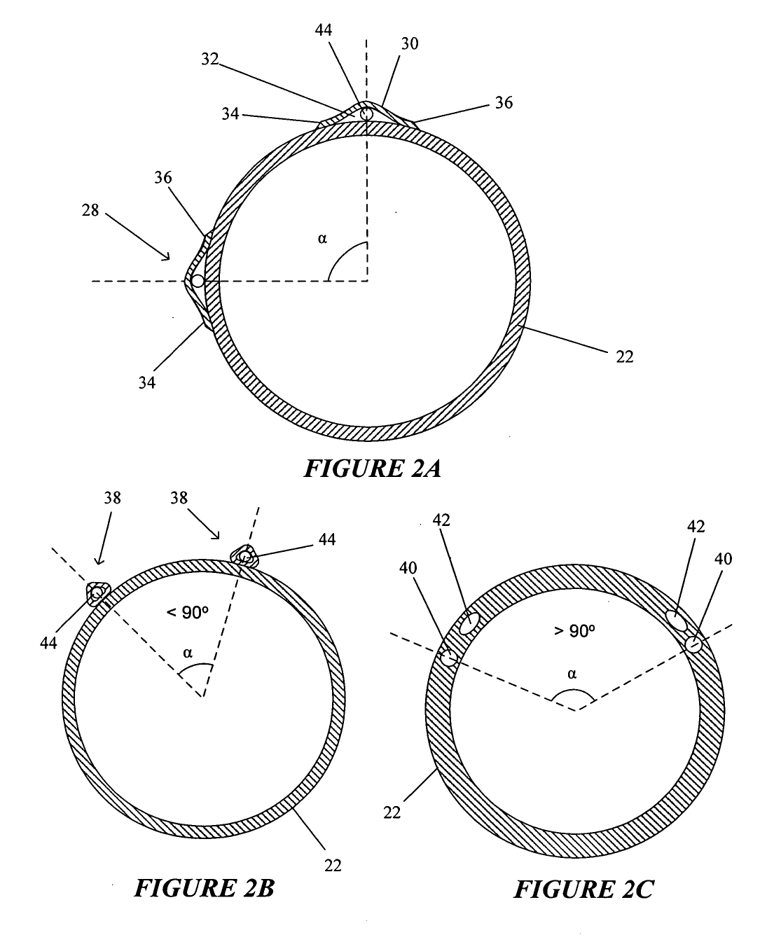

Catheters of the present invention comprise an elongate tubular member defining an inner lumen extending from a proximal end toward a distal end of the tubular member. FIGS. 1 and 2A-2C illustrate a proximal end of a catheter 20 of the present invention comprising a continuous and generally cylindrical side wall 22 forming a lumen 24. Longitudinal channels 28 are formed on or associated with external catheter surface 26 and comprise a channel wall 30 forming a channel lumen 32.

In the embodiment illustrated in FIGS. 1 and 2A, channel 28 is formed by bonding or otherwise affixing two opposite longitudinal sidewalls 34, 36 of a generally rectangular channel wall 30 to the exterior surface 26 of catheter side wall 22, forming channel lumen 32 between the sidewalls that are bonded or affixed to or otherwise associated with the catheter side wall. In an alternative embodiment illustrated in FIG. 2B, external channels 38 may be formed as an integral structure such as a cylinder or a triangular or other polygonal tubular structure, for example, having a wall bonded or otherwise affixed to or associated with catheter side wall 22. In yet another embodiment, illustrated in FIG. 2C, channels 40, 42 may be provided as lumens through the sidewall 22 of catheter 20. Channel lumens are sized to accommodate insertion and/or withdrawal of rod-like stiffener elements through proximal portals.

All or a portion of the internal surfaces of channels 28, 38, 40 and 42 forming the associated channel lumens may be provided with a lubricious surface coating or layer that facilitates sliding of a stiffener member through the channel. Suitable lubricious coatings and materials are known in the art and include, for example, PTFE (TEFLON®) and other fluoropolymer coatings, hydrophilic coatings, and the like.

Where external channels are provided, as illustrated in FIGS. 2A and 2B, the material forming channel wall 30 and external channels 38 is preferably thin, flexible, durable and puncture resistant. The flexibility, bending and pushability profiles of catheters having channels for receiving stiffener elements, as disclosed herein, are preferably substantially similar to the flexibility, bending and pushability profiles of catheters having a similar construction without having stiffener channels. The channel wall(s) may incorporate energy absorbing or viscoelastic polymers that are in a soft and flexible condition at ambient body temperatures and pressures and stiffen with application of a mechanical force, vibration, or the like.

In one embodiment, the composition and/or thickness of the channel wall is substantially constant along the length of each channel, while in another embodiment, the composition and/or thickness of channel wall 30 varies along the length of each channel and is generally more flexible and/or thinner in distal regions of the catheter. Similarly, in some embodiments, the cross-sectional area of each channel lumen may be substantially constant along the length of each channel, while in alternative embodiments, the cross-sectional area and/or dimensions of each channel lumen may vary along the length of each channel. The cross-sectional area of a channel lumen may be reduced in distal regions of the catheter; for example.

In the embodiment illustrated in FIG. 1, the longitudinal axes of channels 28 are generally axially aligned with the longitudinal axis of the catheter sidewall in proximity to the channel and the longitudinal axis of the catheter lumen. Multiple stiffener channels may be provided, as illustrated, and may be arranged in a radially symmetrical or asymmetrical configuration. In the embodiment illustrated in FIG. 2A, two stiffener channels 32 are arranged in proximity to the outer surface of catheter 22 separated by an arc α of about 90°; in the embodiment illustrated in FIG. 2B, two stiffener channels 38 are arranged in proximity to the outer surface of catheter 32 separated by an arc α of less than 90°; and in the embodiment illustrated in FIG. 2C, two pairs of stiffener channels 40, 42 are arranged in the sidewall of catheter 22, with each stiffener channel in a pair being separated from a corresponding stiffener channel in an opposing pair by an arc α of more than 90°.

When multiple stiffener channels are associated with a catheter of the present invention, the dimensions and configurations of each of the stiffener channels may be substantially similar, or stiffener channels having different dimensions and sized to accommodate stiffener elements having different dimensions and/or properties may be provided. This enhances the versatility of the catheter system, since stiffener elements having different properties may be used with a universal catheter depending on the location of the target site within a patient, the tortuosity of the vessels, the interventional device or instrument being guided through the catheter, and the like. Thus, in one embodiment, multiple stiffener channels are provided, each having a different dimension, and multiple stiffener members are provided, each sized to fit in one or more of the channels and having different stiffening properties.

Longitudinal stiffener members 44 are sized for sliding engagement in channel lumens and, in some embodiments, are constructed from a material that is stiffer in the direction of its longitudinal axis than the stiffness of catheter wall 22 in the direction of its longitudinal axis, thereby providing the required additional stabilizing stiffness. Stiffener members 44 according to this embodiment may be provided as rod-like elements that are insertable into and slide through channel lumens after placement of the catheter at a desired target site to stiffen the catheter and enhance the stability of the placement, reducing the risk of catheter movement within a vessel during the use of interventional accessory devices or instruments.

The dimensions and stiffness properties of longitudinal stiffener members 44 may be constant along their lengths or stiffener members 44 may vary in material, construction, cross-sectional area and/or stiffness along their lengths. In general, variable flexibility stiffener elements are less flexible in proximal regions and may be more flexible in distal regions. Stiffener members may have various cross-sectional profiles including generally circular, generally oblong or ovoid, generally triangular with arced corners, and other polygonal configurations. The relative cross-sectional dimensions and profiles of stiffener members for use in catheters having accommodating channel lumens are designed to provide smooth sliding of stiffener members through channel lumens. Stiffener members may have a lubricious coating or outer layer that facilitates sliding of the stiffener members through channel lumens. Similarly, the inner channel wall may have a lubricious coating or layer that facilitates sliding of the stiffener members through the channel lumens. Suitable lubricious coatings and materials are known in the art and include, for example, PTFE (TEFLON®) and other fluoropolymer coatings, hydrophilic coatings, and the like.

In another embodiment, the stiffener members and/or inner channel wall may have surface discontinuities that reduce friction and facilitate sliding of the surfaces in relationship to one another. Surface discontinuities may be provided in the form of “dimples” or relatively shallow disc-shaped depressions. In alternative embodiments, the surface discontinuities may be generally triangular, oval, oblong, provided in curved arcs or serpentine shapes, or in any other configurations that facilitate sliding of stiffener members within the channels. In preferred embodiments, the maximum depth of discontinuities is relatively shallow and is less than about 50μ, while the maximum surface dimension of discontinuities is less than about 100μ and, more preferably, less than about 50μ. The pattern of surface discontinuities is preferably regular, though irregular patterns may be implemented for specific applications. The density and/or pattern and or configuration of discontinuities may vary along the length of a stiffener or channel lumen, with higher density discontinuities in areas of tighter contact to improve sliding of stiffener members within the channel lumen.

In yet another embodiment, surface discontinuities may be provided in the form of grooves that are generally longitudinal or curved or provided in a helical or spiral configuration. Helical grooves may have a constant or variable pitch and may spiral in either a left or right direction, or may comprise sections spiraling in both left and right directions. The grooved inner lumen wall of the catheter may alternatively or additionally comprise lands and grooves in a pattern that facilitates both passage and rotation of a stiffener member through the channel lumen.

The distal tips of rod-like stiffener elements are generally blunt and atraumatic to facilitate sliding within the channel lumen and to prevent punctures to the channel side wall during insertion of the stiffener elements. In one embodiment, rod-like stiffener elements are longer than the length of mating channel lumens so that when the stiffener elements are fully inserted, a length projects from the proximal portal of the stiffener channel to permit withdrawal of the stiffener from the channel. In another embodiment, rod-like stiffener elements have an enlargement or stop or handle at their proximal end that limits insertion of stiffener elements through mating channels and provides a structure for grasping and withdrawing the stiffener elements from the channels. In yet another embodiment, stiffener elements may be provided with stops in the form, for example, of enlargements or mechanical coupling devices that are insertable into mating recesses or other coupling mechanisms provided in accessory devices that remain outside the body to limit insertion of stiffener members into channels. This system provides insertion of stiffener members to selectable lengths or points along the length of the catheter. Stiffener members may be marked at desired distances or locations to indicate distance or location along the catheter.

The stiffener elements may be constructed from a variety of materials. In general, biocompatible metallic, thermoplastic, ceramic and/or cermet materials may be suitable. Suitable materials include stainless steel, nitinol and other nickel-titanium alloys, titanium and titanium alloys. In some embodiments, stiffener elements are preferably constructed from a “shape memory” material, such as a nickel/titanium alloy (optionally containing modest amounts of iron), a copper/zinc alloy optionally containing beryllium, silicon, tin, aluminum or gallium, or a nickel/aluminum alloy. Super elastic nickel titanium alloys known as “nitinol” alloys tolerate significant flexing without plastic deformation, even when used as a very small diameter wire, and are especially preferred for some embodiments.

Stiffener elements may alternatively or additionally be constructed from materials that assume two or more different configurations based on exposure to a shape change condition. In one condition (e.g. ambient body temperature, electrical and magnetic fields), for example, the stiffener members are rod-like and in another, activated condition produced by changing the temperature, applying current, applying an electrical or magnetic field, or the like, the stiffener members adopt a second predetermined conformation in which they are shaped in predetermined locations to stabilize the catheter and prevent movement of the catheter during use of an interventional accessory device or instrument. The shape adopted by portions of stiffener rods following activation may, for example, conform to the path of blood vessels in tortuous sections of the vasculature, such as the aortic arch. In this system, upon activation of the stiffener rods to their shaped configuration, the catheter is effectively lodged in the vasculature and stabilized. Stiffener rods having these properties are also preferably releasable by again changing the temperature, applying current, applying a magnetic field, or the like, so that the rods resume their relaxed, generally rod-like condition for withdrawal after completion of the intervention.

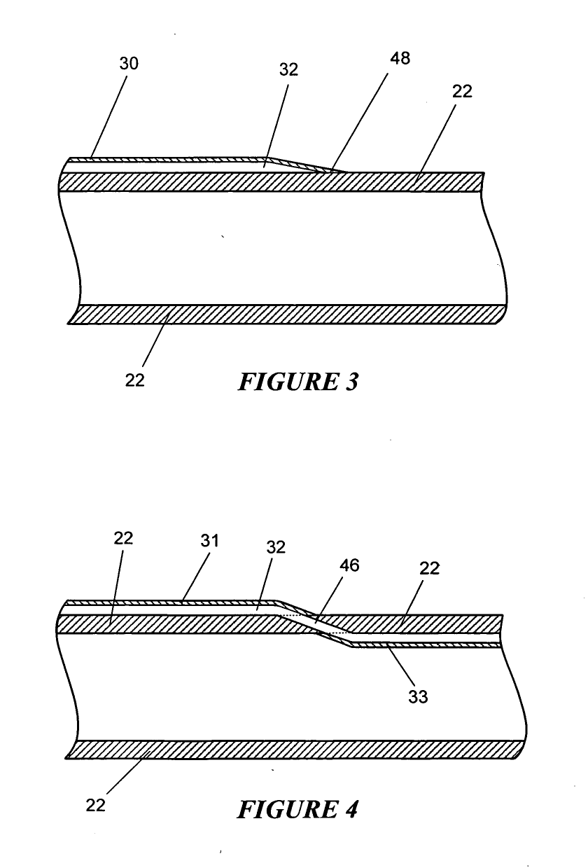

Stiffener channels and stiffener members may extend for substantially the length of the catheter, or the channels may terminate proximal to the distal end of the catheter. In the embodiment illustrated in FIG. 3, for example, channel lumen 32 resides between an outer surface of catheter side wall 22 and channel wall 30. Channel wall 30 is bonded or otherwise affixed to catheter side wall at a channel termination point 48 that is proximal to the distal tip of catheter 20. As a stiffener member is inserted and guided through lumen 32, its forward progress is stopped at termination point 48. Multiple channels provided on or in association with catheter may not only have different dimensions, profiles and stiffness properties, but they may terminate at different points along the length of the catheter. Stiffener members may likewise be provided in different lengths to match the different termination points for different stiffener channels.

FIG. 4 illustrates another embodiment in which a continuous channel lumen 32 traverses catheter sidewall 22 and is located partially in proximity to an outer catheter side wall and partially in proximity to an inner catheter surface. In this embodiment, an external channel sidewall 31 forms an external portion of lumen 32 for a distance along the catheter and an internal channel sidewall 33 forms an internal portion of lumen 32 for a distance along the catheter. The external and internal portions of lumen 32 are continuous by means of passage 46 traversing the sidewall of catheter 20. Passage 46 is preferably angled and gradual to provide a smooth transition between the external and internal portions of lumen 32. The internal portion of lumen 32 may terminate proximal to the distal tip of the catheter, or it may be provided with a distal channel portal in proximity to the distal tip of catheter 20. The distal channel portal allows passage of a distal end of a stiffener member through the portal. Stiffener members for use in connection with a catheter having a distal channel portal in proximity to a distal end of the catheter tip may serve as a primary or secondary guidewire or lead.

While in the foregoing specification this invention has been described in relation to certain preferred embodiments thereof, and many details have been set forth for purposes of illustration, it will be apparent to those skilled in the art that the invention is susceptible to various changes and modification as well as additional embodiments and that certain of the details described herein may be varied considerably without departing from the basic spirit and scope of the invention.

All of the patent references and publications cited in this specification are incorporated by reference herein in their entireties.

We claim:

1. A catheter comprising a generally tubular structure having a substantially continuous side wall forming an outer catheter wall and having primary catheter lumen and additionally comprising at least one longitudinal channel in proximity to the side wall of the catheter, the longitudinal channel being generally coaxial with a longitudinal axis of the primary catheter lumen and having a proximal portal.

2. A catheter of claim 1, wherein the at least one longitudinal channel extends for substantially the full length of the catheter.

3. A catheter of claim 1, wherein the at least one longitudinal channel extends for less than the full length of the catheter.

4. A catheter of claim 1, additionally comprising at least one rod-like stiffening member sized for insertion into and withdrawal from the longitudinal channel.

5. A catheter of claim 4, wherein the rod-like stiffening member is at least partially inserted in the longitudinal channel.

6. A catheter of claim 4, wherein the rod-like stiffening member has a lubricious layer or coating on at least a portion of its outer surface.

7. A catheter of claim 4, wherein the rod-like stiffening member is formed from a shape memory material.

8. A catheter of claim 4, wherein at least one of the stiffening member and an inner wall of the longitudinal channel has surface discontinuities that reduce friction and facilitate sliding of the surfaces in relationship to one another.

9. A catheter of claim 4, wherein the distal tip of the stiffening member is atraumatic.

10. A catheter of claim 4, wherein the length of the stiffener element is greater than the length of the longitudinal channel.

11. A catheter of claim 1, comprising at least two longitudinal channels, wherein the channels are radially separated from one another by about 45-180°.

12. A catheter of claim 1, comprising at least two longitudinal channels, wherein the channels are radially separated from one another by about 60-115°.

13. A catheter of claim 1, comprising at least two longitudinal channels, wherein the channels are radially separated from one another by about 80-100°.

14. A catheter of claim 1, comprising multiple longitudinal channels arranged in a radially asymmetrical configuration.

15. A catheter of claim 1, comprising multiple longitudinal channels having different channel conformations, different channel sizes or different channel lengths.

16. A catheter of claim 1, wherein the longitudinal channel comprising a lubricious layer or coating on an inner channel wall.

17. A catheter of claim 1, wherein the longitudinal channel comprises an energy absorbing or viscoelastic polymer material having variable stiffness properties.
